(19) United States
(12) Patent Application Publication
(10) Pub. No.: US 2023/0130343 A1
(43) Pub. Date: Apr. 27, 2023

(54) Crystal Growing Condition Analysis Method, Crystal Growing Condition Analysis System, Crystal Growing Condition Analysis Program, and Data Structure for Crystal Growing Data

(71) Applicant: Nippon Telegraph and Telephone Corporation, Tokyo (JP)

(72) Inventors: Ryo Nakao, Tokyo (JP); Takuya Otsuka, Tokyo (JP); Tomonari Sato, Tokyo (JP)

(21) Appl. No.: 17/915,398

(22) PCT Filed: Mar. 31, 2020
(86) PCT No.: PCT/JP2020/014860

§ 371 Date: Sep. 28, 2022

(51) Int. Cl. G16C 20/10; G16C 20/70

(52) U.S. Cl. CPC G16C 20/10; G16C 20/70

(57) ABSTRACT

An analysis method of crystal growth conditions includes a step of calculating an evaluation function on the basis of results obtained by measuring crystals grown under varied crystal growth conditions, a step of performing machine learning of the evaluation function, and a step of obtaining optimum crystal growth conditions from a result of the machine learning, wherein the evaluation function is based on a difference between crystal quality data of an ideal crystal and crystal quality data of the crystal having been grown.

### CROSS-REFERENCE TO RELATED APPLICATIONS

This patent application is a national phase filing under section 371 of PCT application no. PCT/JP2020/014860, filed on Mar. 31, 2020, which is incorporated herein by reference in its entirety.

### TECHNICAL FIELD

The present invention relates to an analysis method of crystal growth conditions, an analysis system of crystal growth conditions, an analysis program of crystal growth conditions, and a data structure of crystal growth data, for automatically optimizing crystal growth conditions.

### BACKGROUND

In a crystal growth step for semiconductor crystals and the like, there have been used crystal growth devices of a molecular beam epitaxy (MBE) method, a metalorganic vapor phase epitaxy (MOVPE) method and the like. These crystal growth devices can obtain a desired stacking structure of crystals and thin films of semiconductor and the like by optimizing many crystal growth conditions (parameters) such as the amounts of introduction and the temperature of raw materials used, the speed of growth, and the rotational speed of a substrate.

Patent Literature 1 discloses, as an example, a technology of performing crystal growth with crystal growth conditions optimized.

### CITATION LIST

### Patent Literature

Patent Literature 1: Japanese Patent Laid-Open No. H10-242051.

### SUMMARY

### Technical Problem

However, since these parameters affect one another and their optimum values are different for each crystal growth device, whether an excellent crystal is obtained or not has depended significantly on experiences and techniques of technical experts of crystal growth and much trial and error has been needed.

Since training such technical experts and optimizing the crystal growth conditions (parameters) by trial and error require time and costs, it has been a great problem to optimize the parameters automatically.

### Means for Solving the Problem

In order to solve the problem as mentioned above, there is provided an analysis method of crystal growth conditions according to embodiments of the present invention, including: a step of calculating an evaluation function on the basis of results obtained by measuring crystals grown under varied crystal growth conditions; a step of performing machine learning of the evaluation function; and a step of obtaining optimum crystal growth conditions from a result of the machine learning, wherein the evaluation function is based on a difference between crystal quality data of an ideal crystal and crystal quality data of the grown crystals.

Moreover, there is provided an analysis method of crystal growth conditions according to embodiments of the present invention, including: a step of calculating an evaluation function on the basis of results obtained by measuring crystals grown under varied crystal growth conditions; a step of performing machine learning of the evaluation function; and a step of obtaining optimum crystal growth conditions from a result of the machine learning, wherein the evaluation function is based on a ratio between an intensity and a half-value width of a peak of crystal quality data of the crystals.

Moreover, there is provided an analysis method of crystal growth conditions according to embodiments of the present invention, comprising: a step of calculating an evaluation function on the basis of XRD curves of crystals grown under varied crystal growth conditions; a step of performing machine learning of the evaluation function; and a step of obtaining optimum crystal growth conditions from a result of the machine learning, wherein the evaluation function is based on a ratio between an intensity of a substrate peak and an intensity of a diffraction peak on the XRD curve.

Moreover, there is provided an analysis system of crystal growth conditions according to embodiments of the present invention, comprising: an input unit, a storage unit, an analysis unit, and an output unit, wherein to the input unit, crystal growth conditions and crystal growth data based on crystal quality data obtained by measuring crystals grown under varied crystal growth conditions are input, the storage unit stores the crystal growth data, the analysis unit acquires the crystal growth data from the storage unit, calculates an evaluation function from the crystal growth data, performs machine learning on the crystal growth conditions and the evaluation function, and analyzes optimized crystal growth conditions, and the output unit outputs a result obtained by the analysis.

Moreover, there is provided an analysis program of crystal growth conditions according to embodiments of the present invention for causing an analysis system of crystal growth conditions which analyzes optimized crystal growth conditions to function to perform processing of: calculating an evaluation function from crystal growth conditions and crystal growth data based on crystal quality data obtained by measuring crystals grown under varied crystal growth conditions; performing machine learning on the crystal growth conditions and the evaluation function; and analyzing optimized crystal growth conditions.

Moreover, there is provided a data structure of crystal growth data according to embodiments of the present invention, being used for an analysis system of crystal growth conditions including an input unit, a storage unit, an analysis unit, and an output unit, the data structure of crystal growth data being stored in the storage unit, the data structure comprising: a crystal growth condition item ID indicating an item of crystal growth conditions; crystal growth condition data indicating a crystal growth condition value at the crystal growth condition item ID; and crystal evaluation data which makes a pair together with the crystal growth condition data, the data structure being used for processing of: selecting the crystal growth condition item ID in the storage unit; and the analysis unit acquiring, for the selected crystal growth condition item ID, the crystal growth condition data and the crystal evaluation data that makes a pair together with the relevant crystal growth condition data, from the storage unit, to calculate an evaluation function, analyzing the evaluation function by machine learning, and analyzing optimized crystal growth conditions.

### Effects of Embodiments of the Invention

According to embodiments of the present invention, there can be provided an analysis method of crystal growth conditions, an analysis system of crystal growth conditions, an analysis program of crystal growth conditions, and a data structure of crystal growth data capable of automatically optimizing crystal growth conditions, and time, costs and the like in a crystal growth step can be reduced.

### BRIEF DESCRIPTION OF THE DRAWINGS

FIG. 1 is a configuration diagram of an analysis system of crystal growth conditions according to a first embodiment.

FIG. 2 is a flowchart diagram of the analysis method of crystal growth conditions according to the first embodiment.

FIG. 3 is a diagram showing X-ray diffraction (XRD) curves for explaining the analysis method of crystal growth conditions according to the first embodiment.

FIG. 4 is a diagram for explaining derivation of a parameter in the analysis method of crystal growth conditions according to the first embodiment.

FIG. 5 is a diagram for explaining derivation of the parameter in the analysis method of crystal growth conditions according to the first embodiment.

FIG. 6 is a diagram for explaining derivation of the parameter in the analysis method of crystal growth conditions according to the first embodiment.

FIG. 7 is a diagram for explaining derivation of the parameter in the analysis method of crystal growth conditions according to the first embodiment.

FIG. 8 is a diagram for explaining derivation of the parameter in the analysis method of crystal growth conditions according to the first embodiment.

FIG. 9 is a diagram for explaining derivation of the parameter in the analysis method of crystal growth conditions according to the first embodiment.

FIG. 10 is a diagram showing X-ray diffraction (XRD) curves for explaining an analysis method of crystal growth conditions according to a second embodiment.

FIG. 11 is a flowchart diagram of an analysis method of crystal growth conditions according to a third embodiment.

FIG. 12 is a diagram showing a photoluminescence (PL) spectrum for explaining an analysis method of crystal growth conditions according to a fourth embodiment.

FIG. 13 is a sequence chart showing a series of flows of feeding raw material gases in crystal growth in an example of the analysis method of crystal growth conditions according to an embodiment according to the present invention.

FIG. 14 is a diagram for explaining derivation of a parameter in an example of the analysis method of crystal growth conditions according to an embodiment according to the present invention.

FIG. 15 is a diagram for explaining derivation of the parameter in an example of the analysis method of crystal growth conditions according to an embodiment according to the present invention.

FIG. 16 is a diagram for explaining derivation of the parameter in an example of the analysis method of crystal growth conditions according to an embodiment according to the present invention.

FIG. 17 is a diagram for explaining derivation of the parameter in an example of the analysis method of crystal growth conditions according to an embodiment according to the present invention.

FIG. 18 is a diagram for explaining derivation of the parameter in an example of the analysis method of crystal growth conditions according to an embodiment according to the present invention.

FIG. 19 shows XRD curves of superlattice crystals in an example of the analysis method of crystal growth conditions according to an embodiment according to the present invention.

FIG. 20 is a diagram showing an example of a hardware configuration for an analysis device of crystal growth conditions according to an embodiment according to the present invention.

### DETAILED DESCRIPTION OF ILLUSTRATIVE EMBODIMENTS

### First Embodiment

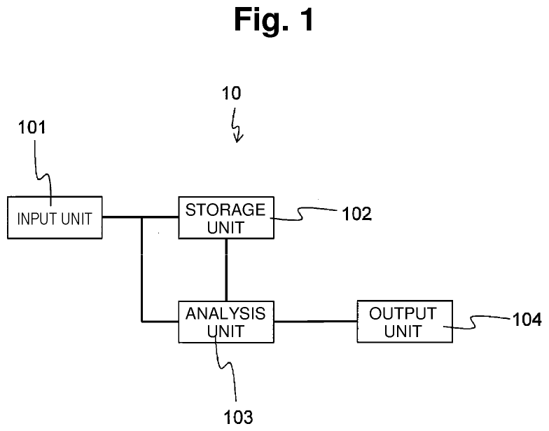

An analysis system of crystal growth conditions and an analysis method of crystal growth conditions according to a first embodiment of the present invention are described with reference to FIGS. 1 to 9.

### Configuration of Analysis System of Crystal Growth Conditions

FIG. 1 shows a configuration of an analysis system 10 of crystal growth conditions according to the present embodiment. The analysis system 10 of crystal growth conditions includes an input unit 101, a storage unit 102, an analysis unit 103 and an output unit 104. Crystal growth data is input to the input unit 101. The input data is stored in the storage unit 102. The analysis unit 103 acquires the crystal growth data from the storage unit 102 and analyzes optimization conditions for crystal growth. The analyzed optimization conditions are output to the output unit 104.

### Analysis Method of Crystal Growth Conditions

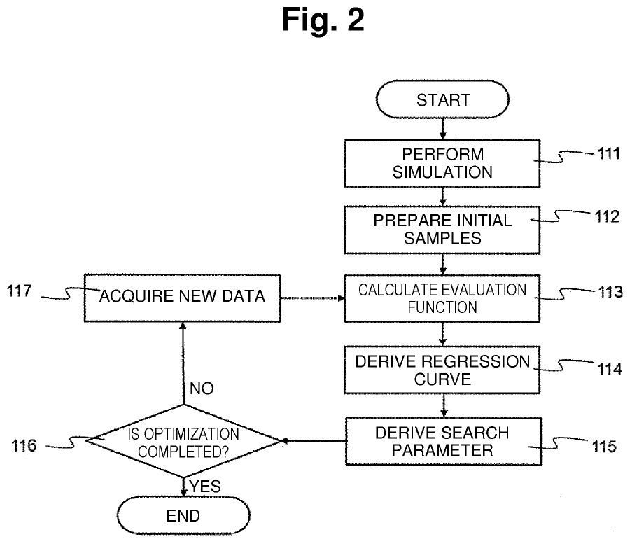

FIG. 2 shows a flowchart of an analysis method of crystal growth conditions according to the present embodiment. In this analysis method, X-ray diffraction (hereinafter referred to as “XRD”) is used as a technique of evaluating crystallinity.

In the analysis method of crystal growth conditions according to the present embodiment, an XRD curve obtained by XRD is used as data indicating crystal quality (hereinafter referred to as “crystal quality data”). An example of the present embodiment is described using a semiconductor superlattice structure for the crystal structure.

First, in order to acquire an XRD curve of an ideal crystal in XRD measurement, the structure of the ideal crystal is simulated (step 111). While a technique of simulating XRD can be performed, for example, with a simulator or the like bundled with analysis software provided by the manufacturer of an XRD device, it may be performed in another way.

Next, initial sample crystals are prepared (step 112). Specifically, a plurality of sample crystals are caused to grow under a plurality of sets of crystal growth conditions (parameters).

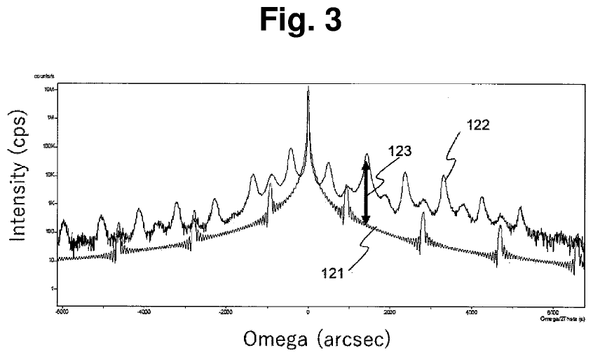

Next, an XRD measurement is performed for each sample crystal grown with the corresponding set of parameters. FIG. 3 shows an XRD curve 121 of the ideal crystal obtained by the simulation, and an XRD curve 122 of the sample crystal actually grown. Since the conditions (parameters) of the sample crystal are not optimized, a difference 123 arises between the XRD curve 121 of the ideal crystal and the XRD curve 122 of the sample crystal. Such measurement and simulation of XRD are used, also in ordinary crystal growth, for the purpose of evaluating crystal quality, and they do not raise time and economical costs.

Next, an evaluation function is calculated for the XRD curve (step 113). The evaluation function (hereinafter referred to as “EF”) is herein set to be the difference 123 between the XRD curve 121 of the ideal crystal and the XRD curve 122 of the sample crystal. Since the EF represents a divergence of the sample crystal from the ideal crystal, to derive the parameters that minimize the EF is equivalent to searching for the parameters that afford the optimized crystal structure.

Specifically, the EF can be defined by the following Expression (1), where ε(ω) denotes the difference from the ideal crystal at each measurement point (diffraction angle) ω in the XRD measurement.

EF(XRD)1=∫ε(ω)dω Expression (1):

While the evaluation function is herein determined for the difference in intensity, the evaluation function may be determined using a deviation of the diffraction angle ω or the like. As above, the evaluation function is calculated using the XRD curve as the crystal quality data.

Next, a graph is created in which the axis of ordinates represents the value of the evaluation function EF and the axis of abscissas represents the value of a parameter, and a regression curve is derived (step 114) to derive a parameter (hereinafter referred to as “search parameter”) to be searched in the next analysis (step 115). For example, Gaussian process regression can be used for deriving the regression curve. A technique of deriving the regression curve is not limited thereto, but otherwise, a least squares method using an exponential function, a quadratic function or the like, and the like can be used.

Otherwise, an expected improvement (hereinafter referred to as “EI”) can be acquired as Bayesian optimization. In the Bayesian optimization, the optimum point can be automatically searched for by setting the parameter point where the EI is at its maximum to the next search point. The Bayesian optimization is a technique which can efficiently obtain the parameter to be next searched even with a few data points, and effectively works for a step which raises time and economical costs, such as crystal growth (Kota Matsui, et al. “Introduction to Bayesian optimization and Its Application to Material Engineering”, Materia (Materia Japan), Vol. 58, No. 1 (2019)).

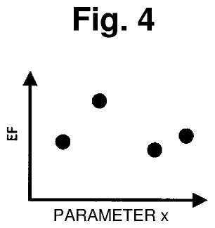

FIGS. 4 to 9 show an example of deriving the search parameter using the Gaussian process regression and the Bayesian optimization.

First, EF(XRD)1 is calculated using Expression (1) on the basis of XRD curves of four sample crystals having grown under four sets of conditions, and a graph is created, where the axis of ordinates represents the value of the evaluation function EF and the axis of abscissas represents the value of the parameter (FIG. 4; step 113).

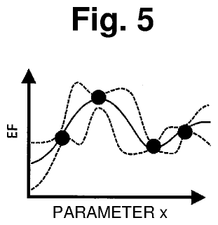

Next, a regression curve is derived using the Gaussian process regression. In the figure, the solid line represents the average value and the dotted lines represent the maximum value and the minimum value of the variance (FIG. 5; step 114).

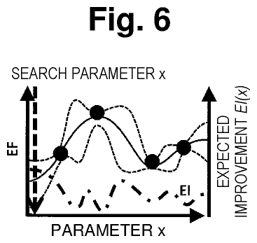

Next, a search parameter is derived (FIG. 6; step 115). In FIG. 6, the dotted arrow indicates the search parameter. Moreover, in FIG. 6, the dash and dot line represents the EI obtained by the Bayesian optimization.

In the present embodiment, as a candidate for the search parameter, that is, the optimization condition, the parameter is selected the average of which presents the minimum value in the regression curve of the EF. Otherwise, since a region that has a large width (difference between the maximum value and the minimum value) of a variance provides a wide range within which a condition can be selected and a high possibility that the optimum condition exists therein, a parameter can also be selected that presents the maximum value of the width of the variance in the regression curve of the EF. Otherwise, a parameter can also be selected that presents the maximum value of the EI.

Next, the completion of the optimization is determined (step 116). For example, when the value of the EF or the EI in the search parameter reaches a predefined value, the optimization completes to finish the analysis. Otherwise, the condition for determining the completion of the optimization may be the case of reaching a predefined number of times of calculation, the case where the variance value reaches a predefined value, or the similar case. As above, it is determined, with a predetermined condition, whether the optimization is to complete, leading to the analysis, or is to be continued.

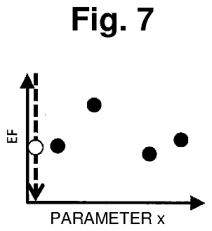

When it is determined that the optimization is not completed, crystal growth is performed with the search parameter derived in step 115, and a sample crystal caused to grow is measured to acquire new data (step 117). FIG. 7 shows the acquired new data by the open circle.

The steps from the calculation of the evaluation function (step 113) are performed again. First, from the new data, an evaluation function EF is calculated (FIG. 7; step 113). FIG. 7 shows the evaluation function EF calculated from the new data by the open circle.

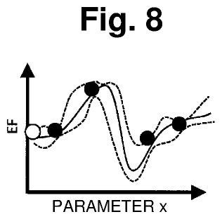

Next, a regression curve is derived using the Gaussian process regression (FIG. 8; step 114).

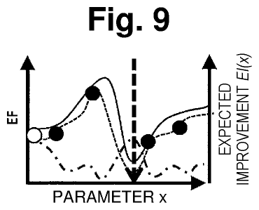

Next, a search parameter is derived (FIG. 9; step 115). In FIG. 9, the dotted line arrow indicates the search parameter. Moreover, in FIG. 9, the dash and dot line represents the EI obtained by the Bayesian optimization. The analysis process above is repeated until the determination result of the completion of the optimization is obtained by reaching the predetermined condition as mentioned above.

As above, the optimum crystal growth conditions can be automatically analyzed by performing machine learning on crystal growth conditions and evaluation functions (EF, EI and/or the like) which are paired crystal growth data according to the present embodiment. As a result, time, costs, human resources and the like in a crystal growth step can be reduced.

When a regression curve by a least squares method is used other than the Gaussian process regression, a point which minimizes this regression curve can also be set as the next search condition. As above, by repeating the procedure of these until the condition optimization completes, the conditions can be optimized with no need for experiences and knowledge of technical experts.

### Second Embodiment

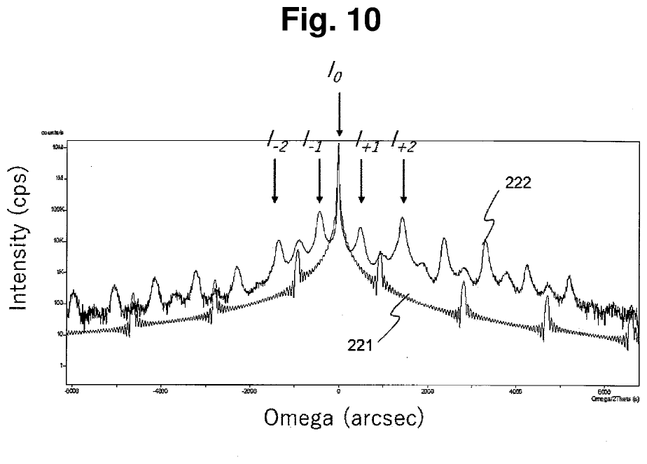

An analysis system of crystal growth conditions and an analysis method of crystal growth conditions according to a second embodiment of the present invention are described with reference to FIG. 10. A configuration of the analysis system of crystal growth conditions according to the present embodiment is similar to that in the first embodiment. The present embodiment is different from the first embodiment in the analysis method of crystal growth conditions.

### Analysis Method of Crystal Growth Conditions

In the analysis method of crystal growth conditions according to the present embodiment, there is set, as the object to be evaluated, a deviation of the diffraction angle co from the structure of the ideal crystal on the XRD curve, and the analysis is performed using an mth-order diffraction peak intensity Im, which does not appear with the structure of an ideal crystal.

FIG. 10 shows the XRD curve 221 of the ideal crystal by the simulation, and the XRD curve 222 of the sample crystal actually grown. On the curve 222 of the sample crystal actually grown, an mth-order diffraction peak (m is an integer) is observed, which does not appear on the curve 221 of the ideal crystal.

The evaluation function EF is calculated with the following Expression (2) using the intensity Im of this mth-order diffraction peak and an intensity Io of a oth-order peak (hereinafter referred to as “substrate peak”) on the curve 222 of the sample crystal. Here, since Io is a peak from a substrate, it appears on any of the simulation result and the result of the sample crystal actually grown. Therefore, Io is supposed not to be included when Im is accumulated.

$$E F ( X R D ) 2 = - {log}_{10} \frac{{\sum }_{N} {I}_{\pm m}}{{I}_{0}} \qquad Expression ( 2 )$$

Since this evaluation function EF(XRD)2 indicates that the structure is closer to that of the ideal crystal as the value is larger, the value, of this evaluation function, becoming larger indicates the optimization advancing. While the accumulation is performed up to the Nth-order diffraction peak, its degree may be properly selected depending on the extent of influence on the system targeted.

Otherwise, when the deviation of the diffraction angle ω from the structure of the ideal crystal on the XRD curve is set as the object to be evaluated, the evaluation may be performed using the difference between the XRD curve of the ideal crystal obtained by the simulation and the XRD curve of the sample crystal as with the first embodiment.

As above, using EF(XRD)2 obtained using the XRD curve as the crystal quality data, the crystal growth conditions are optimized with the regression curves and the Bayesian optimization as with the first embodiment.

According to the analysis method of crystal growth conditions according to the present embodiment, the optimum crystal growth conditions can be automatically analyzed by performing machine learning on crystal growth conditions and evaluation functions (EF, EI and/or the like) which are paired crystal growth data, and time, costs, human resources and the like in a crystal growth step can be reduced.

### Third Embodiment

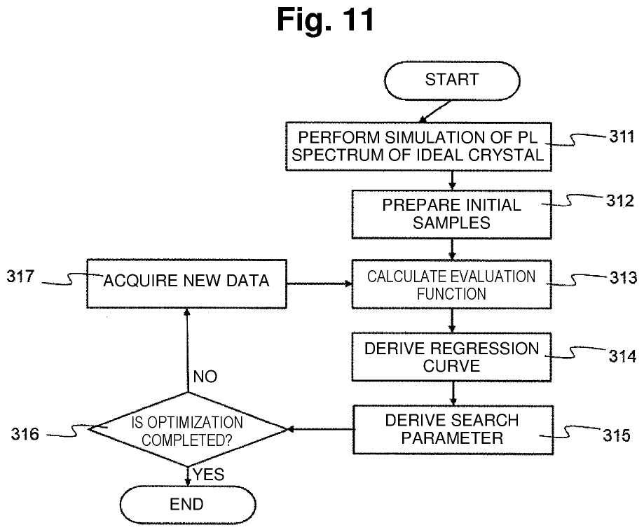

An analysis system of crystal growth conditions and an analysis method of crystal growth conditions according to a third embodiment of the present invention are described with reference to FIG. 11. A configuration of the analysis system of crystal growth conditions according to the present embodiment is similar to that in the first embodiment. The present embodiment is different from the first and second embodiments in the analysis method of crystal growth conditions.

### Analysis Method of Crystal Growth Conditions

In the analysis method of crystal growth conditions according to the present embodiment, analysis is performed using photoluminescence measurement (hereinafter referred to as “PL”). In evaluation of a crystal, it is also important to evaluate characteristics of PL, for example, for a light emitting material or the like. Therefore, in the present embodiment, the crystal growth conditions (parameters) are optimized from a regression curve or the like by using PL spectra as crystal quality data and calculating an evaluation function EF(PL) with the following Expression (3) from the difference between a PL spectrum obtained by simulation and a PL spectrum of a sample crystal actually grown. Herein, λ is a PL wavelength.

EF(PL)1=∫ε(ω)dω Expression (3):

FIG. 11 shows a flowchart diagram of the analysis method of crystal growth conditions according to the present embodiment. The analysis can be performed in the similar process to that in the first embodiment except that the crystal quality data used for the analysis are PL spectra.

According to the analysis method of crystal growth conditions according to the present embodiment, the optimum crystal growth conditions can be automatically analyzed by performing machine learning on crystal growth conditions and evaluation functions (EF, EI and/or the like) which are paired crystal growth data (steps 311-317), and time, costs, human resources and the like in a crystal growth step can be reduced.

### Fourth Embodiment

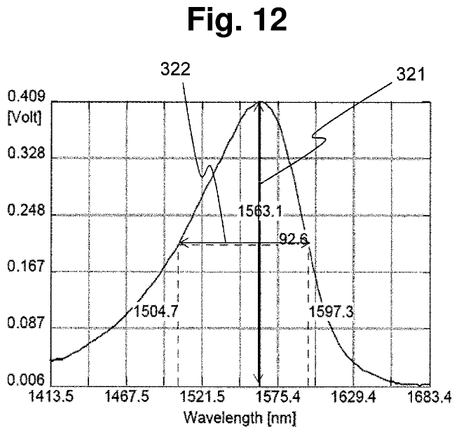

An analysis system of crystal growth conditions and an analysis method of crystal growth conditions according to a fourth embodiment of the present invention are described with reference to FIG. 12.

A configuration of the analysis system of crystal growth conditions according to the present embodiment is similar to that in the first embodiment. The present embodiment is different from the first to third embodiments in the analysis method of crystal growth conditions.

### Analysis Method of Crystal Growth Conditions

In the analysis method of crystal growth conditions according to the present embodiment, analysis is performed using PL spectra obtained by PL measurement as the crystal quality data. FIG. 12 shows an example of a peak in a PL spectrum of a sample crystal actually grown. In the present embodiment, an evaluation function EF(PL)2 based on a PL spectrum is calculated with the following Expression (4) using the intensity of a peak in the PL spectrum (hereinafter referred to as “PL intensity”) 321 and the half-value width thereof (hereinafter referred to as “PL half-value width”) 322.

$$EF ( P L ) 2 = \frac{PL Intensity}{PL Half - Value Width} \qquad Expression ( 4 )$$

This is because a light emitting material is typically represented more by an ideal crystal structure for a higher intensity of light emission and a narrower half-value width of light emission, it is indicated to be closer to the structure of the ideal crystal as this value is larger. It should be noted that since in the case of a material which is intended to emit light with a wide bandwidth, the half-value width of PL is better as being wider, it is desired to be placed at the numerator of the evaluation function, not at the denominator thereof. Otherwise, only the PL intensity or only the half-value width may be used, or the wavelength of light emission may be used, for example.

While in the present embodiment, the ratio between the intensity of a peak in a PL spectrum obtained by PL measurement and the half-value width thereof is used, the ratio between the intensity of a peak in crystal quality data obtained by other measurement such as an XRD curve and the half-value width thereof may be used.

Using EF(PL)2 obtained as above, the crystal growth conditions are optimized with the regression curve and the Bayesian optimization as with the first embodiment.

According to the analysis method of crystal growth conditions according to the present embodiment, the optimum crystal growth conditions can be automatically analyzed by performing machine learning on crystal growth conditions and evaluation functions (EF, EI and/or the like) which are paired crystal growth data, and time, costs, human resources and the like in a crystal growth step can be reduced.

### Fifth Embodiment

An analysis system of crystal growth conditions and an analysis method of crystal growth conditions according to a fifth embodiment of the present invention are described. A configuration of the analysis system of crystal growth conditions according to the present embodiment is similar to that in the first embodiment. As to the analysis of crystal growth conditions according to the present embodiment, evaluation functions obtained by the analyses of crystal growth conditions according to the first to fourth embodiments are combined and used for the analysis.

### Analysis Method of Crystal Growth Conditions

In the first and second embodiments, the evaluation functions EF(XRD)1 and EF(XRD)2 are calculated using XRD curves as the crystal quality data, and in the third and fourth embodiments, the evaluation functions EF(PL)1 and EF(PL)2 are calculated using PL spectra as the crystal quality data. In the present embodiment, these evaluation functions EF(XRD)1, EF(XRD)2, EF(PL)1 and EF(PL)2 are combined and set as an evaluation function.

Here, each of EF(XRD)2 and EF(PL)2 indicates that the larger its value is, the higher quality the crystal has. On the other hand, each of EF(XRD)1 and EF(PL)1 indicates that the smaller its value is, the higher quality the crystal has. Therefore, when evaluation functions are combined, it is supposed that the reciprocal value of each of EF(XRD)1 and EF(PL)1 is used to indicate that the larger the value of the combined evaluation function is, the higher quality the crystal has.

In the present embodiment, evaluation functions are combined by multiplying the values of the evaluation functions, and the following Expressions (5)-(8) can be used, for example.

EF=1/EF(XRD)1×1/EF(PL)1 Expression (i):

EF=EF(XRD)2×EF(PL)2 Expression (6):

EF=1/EF(XRD)1×EF(XRD)2×EF(PL)2 Expression (7):

EF=1/EF(XRD)1×EF(XRD)2×1/EF(PL)1×EF(PL)2 Expression (8):

The calculation formula for the evaluation function is not limited to these but evaluation functions may be combined for it. Moreover, while in the present embodiment, it is supposed that using the reciprocal values of EF(XRD)1 and EF(PL)1, the larger the value of the combined evaluation function is, the higher quality the crystal has, it may be supposed that using the reciprocal values of EF(XRD)2 and EF(PL)2, the smaller the value of the combined evaluation function is, the higher quality the crystal has. Moreover, while the values of the evaluation functions are multiplied for combining the evaluation functions, they may undergo addition, subtraction and/or division.

As above, using the evaluation function EF obtained based on the crystal quality data, the crystal growth conditions are optimized with the regression curve and the Bayesian optimization as with the first embodiment.

According to the analysis method of crystal growth conditions according to the present embodiment, since the XRD measurement evaluates the crystal structure and the PL measurement evaluates the optical properties of the crystal, a combination of the evaluation functions for both of those can optimize the growth conditions with the crystal multifacetedly evaluated.

Moreover, according to the analysis method of crystal growth conditions according to the present embodiment, the optimum crystal growth conditions can be automatically analyzed by performing machine learning on crystal growth conditions and evaluation functions (EF, EI and/or the like) which are paired crystal growth data, and time, costs, human resources and the like in a crystal growth step can be reduced.

### Specific Example of Analysis Method of Crystal Growth Conditions

There is presented a specific example of optimizing the crystal growth conditions using the analysis method of crystal growth conditions according to the present embodiment. There is used, in this specific example, Expression (6) with EF(XRD)2 obtained in the second embodiment and EF(PL)2 obtained in the fourth embodiment.

For the sample crystal, there was used a superlattice crystal composed of 10 InGaAs well layers and 10 InP barrier layers caused to grow on an InP buffer layer on an InP substrate.

This superlattice crystal was caused to grow using trimethyl indium (hereinafter referred to as “TMI”) and triethyl gallium (hereinafter “TEGa”) as group III raw material gases, and arsine (hereinafter referred to as “AsH3”) and phosphine (hereinafter referred to as “PH3”) as group V raw material gases, for raw material gases, in a hydrogen atmosphere at 620° C.

In order to obtain a high quality crystal through growth of this superlattice crystal, the interface between the InGaAs well layer and the InP barrier layer needs to be a high quality crystal.

One of the crystal growth conditions (parameters) required for causing the crystal the interface of which between the InGaAs well layer and the InP barrier layer is high quality to grow is a condition regarding switching the flow rates of the raw material gases. Specifically, a time period during which each raw material gas is fed and timing when it is fed need to be optimized.

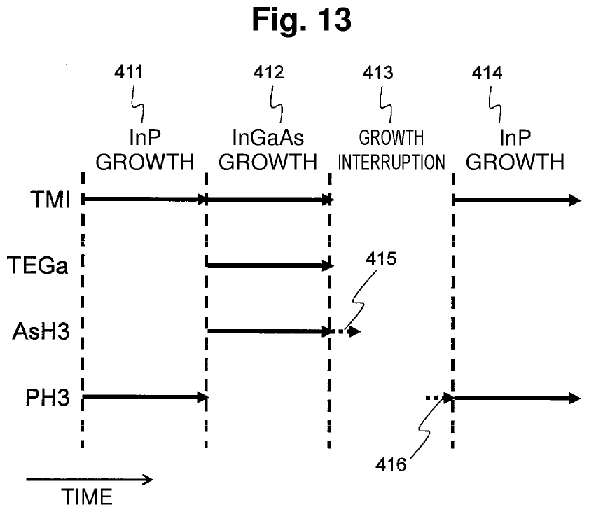

FIG. 13 shows the timing when each raw material gas is fed, being a sequence chart showing a series of flows of feeding the raw material gases in the growth of the superlattice crystal. FIG. 13 shows the flows for causing an InGaAs well layer to grow on an InP barrier layer, and after that, causing an InP barrier layer to grow.

In this specific example, focusing on formation of a growth interface where an InP barrier layer is caused to grow after causing an InGaAs well layer to grow, time periods when the raw material gases are fed during a growth interruption time which is after causing the InGaAs well layer to grow and before causing the InP barrier layer to grow are optimized as crystal growth conditions to be optimized.

First, when causing InP to grow, TMI and PH3 are fed (process 411).

Next, when causing InGaAs to grow, TMI, TEGa and AsH3 are fed (process 412). For the growth of the crystal, both the group III raw material gas(es) and the group V raw material gas(es) need to be fed. Accordingly, the crystal is not caused to grow when feeding only the group V raw material gas(es).

Next, after feeding the raw material gases is stopped to interrupt the growth of the crystal (process 413), TMI and PH3 are fed to cause InP to grow (process 414).

As above, the time for interrupting the growth is provided when InP is to be caused to grow on InGaAs. If the growth interruption time is not provided, this causes contamination with residual gas of the raw material gases (TMI, TEGa and AsH3) at formation of the interface (InP growth) in switching the raw material gases, and hence, the composition of the crystal at the interface does not steeply change, which disables a high quality interface of the crystal to be obtained.

On the other hand, by providing the growth interruption time, the residual gas can be removed and the steep and high quality interface of the crystal without contamination of the residual gas can be obtained. Nevertheless, too long of interruption time causes the crystal surface before the growth (InGaAs surface in this specific example) to be exposed into the hydrogen atmosphere at high temperature, which results in elimination of surface atoms and deterioration of the crystal surface (InGaAs surface in this specific example).

If a crystal (InP in this specific example) is caused to regrow on this deteriorated crystal surface, crystal quality at the interface (InGaAs/InP interface in this specific example) is to deteriorate. As above, feed of the raw material gases during the interruption time is one of the important crystal growth conditions.

In this specific example, optimization of the crystal growth conditions was performed by optimizing a time 415 for feeding AsH3 after stopping the growth of InGaAs (hereinafter referred to as “AsH3 time”) and a time 416 for feeding PH3 before starting InP crystal growth (hereinafter referred to as “PH3 time”) during the growth interruption time.

First, four sample crystals were prepared through crystal growth with the AsH3 time set constant to be a period of 2 seconds and with the PH3 time varied for four conditions (periods of −5, 2, 5 and 8 seconds). Herein, the PH3 time (period of −5 seconds) taking a negative value means that TMI is fed before feeding PH3.

The EF was calculated with Expression (6) using EF(XRD)2 obtained from XRD curves and EF(PL)2 obtained from PL spectra which were measured for these four sample crystals. The regression curve was derived from this EF to search for the optimized crystal growth conditions. Moreover, the EI was evaluated by Bayesian statistics processing to search for the optimized crystal growth conditions.

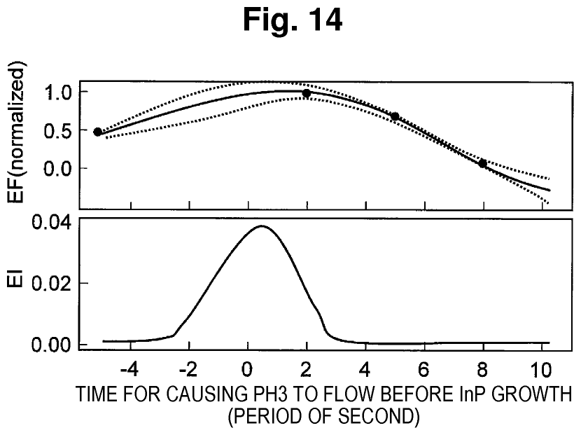

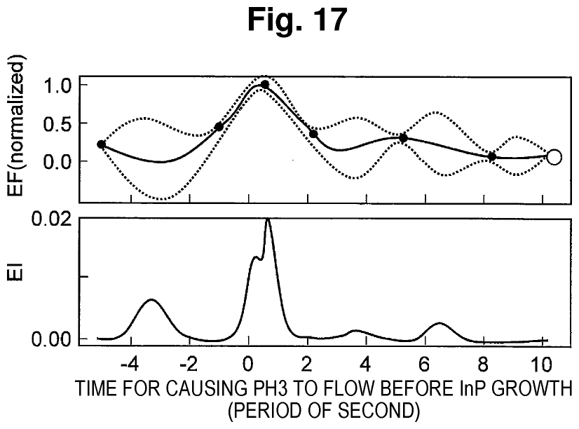

FIG. 14 to FIG. 18 shows a process where the EF and the EI were optimized.

FIG. 14 shows the regression curve of the EF and the acquisition function of the EI with the four sample crystals initially prepared. For the regression curve of the EF, the solid line represents the average, and the dotted lines represent the variance. From FIG. 14, a period of 0.2 seconds of the PH3 time at which the EI shows its maximum value is set to a search parameter. Moreover, at the period of 0.2 seconds of the PH3 time, the variant of the regression curve of the EF is large, and this implies a wide range of selection for the conditions and a high possibility of leading to the optimized conditions.

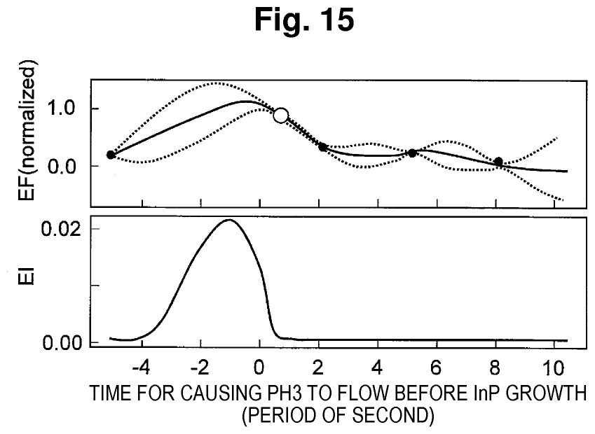

FIG. 15 shows the results of analysis together with the EF (indicated by the open circle in the figure) and the EI of a sample crystal having grown at the period of 0.2 seconds of the PH3 time. From FIG. 15, a period of −1 second of the PH3 time at which the mean of the EF and the EI show their maximum values is set to a search parameter.

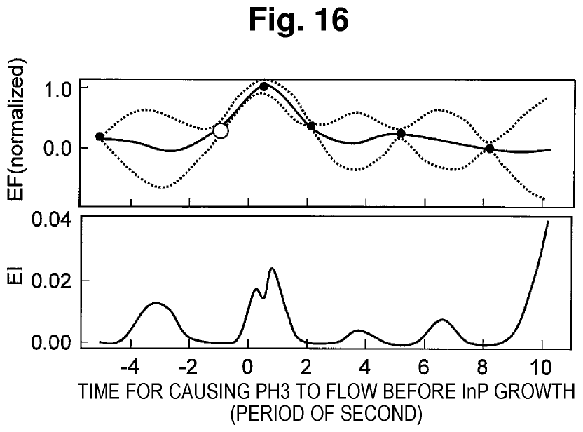

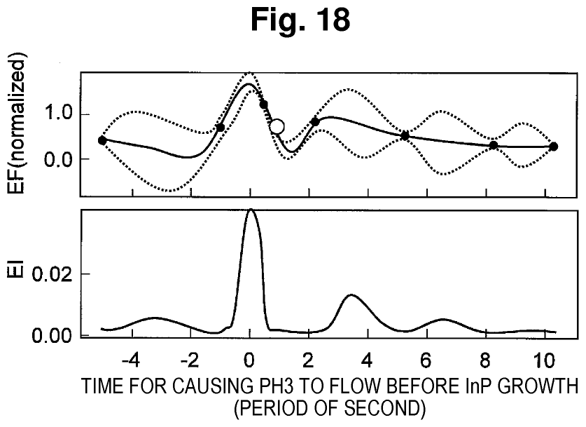

The aforementioned process is repeated as shown in FIG. 16 to FIG. 18 to determine search parameters. From FIG. 18, a period of 0.1 seconds of the PH3 time at which the EI showed its maximum value was determined to be the optimized condition thereof.

Likewise, crystal growth was next performed with the PH3 time set constant to be the period of 0.1 seconds and with the AH3 time varied to perform XRD measurement and PL measurement to evaluate the EF and the EI. As a result, the AsH3 time was optimized to be a period of 0.7 seconds. As above, the crystal growth conditions (parameters) regarding the growth interruption time after the InGaAs crystal growth were optimized to be the period of 0.1 seconds of the PH3 time and the period of 0.7 seconds of the AsH3 time.

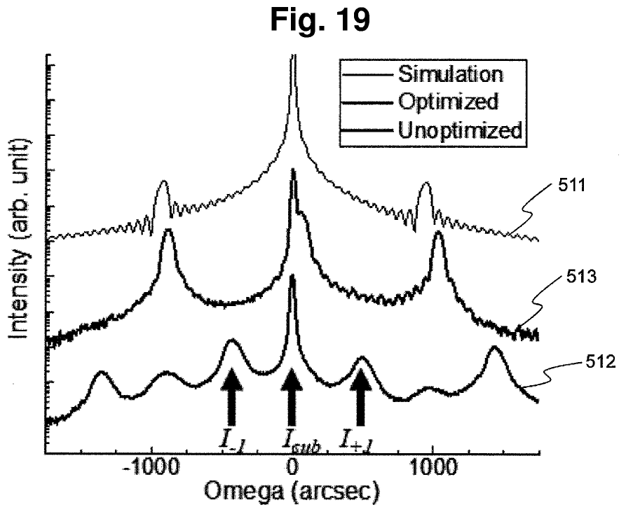

FIG. 19 shows an XRD measurement result of a crystal obtained by the analysis method of crystal growth conditions according to this specific example. The figure shows an XRD curve 511 of an ideal crystal obtained by simulation, an XRD curve 512 of a crystal before optimization, and an XRD curve 513 of a crystal caused to grow under the optimized conditions.

On the XRD curve 512 of the crystal before optimization, peaks are observed at several places as compared with the XRD curve 511 of the ideal crystal. These peaks show the crystal has low quality.

While, on the XRD curve 513 of the crystal grown under optimized condition, peaks observed in the curve 512 are not observed, a curve with substantially the same shape as the XRD curve 511 of an ideal crystal is obtained. This means that high quality crystal was obtained with an optimized condition.

As above, the crystal growth conditions can be optimized by the analysis method of crystal growth conditions according to this specific example. This can result in reduction of time, costs, human resources and the like in a crystal growth step.

Since both the XRD measurement and the PL measurement used in the embodiments according to the present invention are basic techniques for evaluating a crystal, these measurements do not impair productivity. Therefore, the optimum crystal growth conditions can be automatically searched for (machine learned) without productivity deteriorating and with no need for human resources.

While in this specific example, there are used, for a data structure of crystal growth data, the PH3 time and the AsH3 time as crystal growth condition item IDs indicating items (parameters) of the crystal growth conditions, each PH3 time and each AsH3 time which are set to periods of −5 to 10 seconds as crystal growth condition data indicating crystal growth condition values at the crystal growth condition item IDs, and the EF and the EI for each of the PH3 time and the AsH3 time as crystal evaluation data which makes a pair together with the crystal growth condition data, a data structure of crystal growth data with other crystal growth conditions such as growth temperatures and flow rates of raw material gases may be used. There may be employed a data structure of crystal growth data for calculating an evaluation function from the crystal growth condition data at a selected crystal growth condition item ID and the crystal evaluation data which makes a pair together with the crystal growth condition data, and analyzing optimized crystal growth conditions through machine learning of this evaluation function.

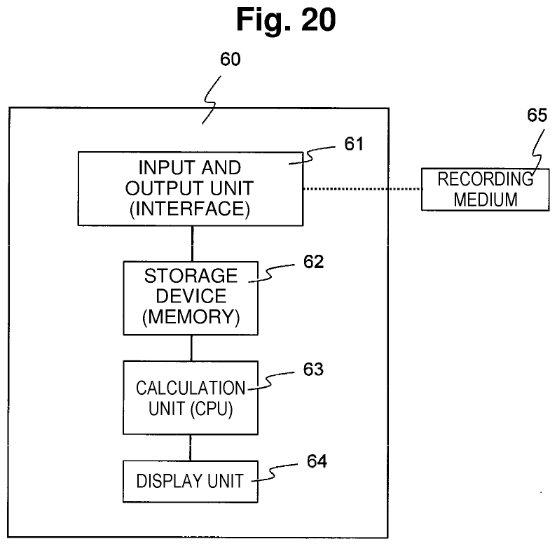

As shown in FIG. 20, the analysis system of crystal growth conditions according to an embodiment of the present invention can be realized by a computer 60 including a display unit 64, a CPU (Central Processing Unit) 63, a storage device 62 and an interface 61, and a program which controls these hardware resources.

In the analysis system of crystal growth conditions according to an embodiment of the present invention, the computer 60 may be included inside the device or may be realized using an external computer. Likewise, the storage unit may use a storage medium 65 outside the device and may read and execute a measurement program stored in the storage medium 65. Storage media includes various magnetic recording media, magnetooptical recording media, a CD-ROM, a CD-R, and various memories. Moreover, an analysis program may be supplied to the computer via a communication line such as the Internet.

The analysis system of crystal growth conditions, the analysis program of crystal growth conditions, and the data structure of crystal growth data according to an embodiment of the present invention also achieve the similar effects to the effects of the analysis method of crystal growth conditions according to an embodiment of the present invention.

While in the first and third embodiments, the XRD curves and PL spectra are used, respectively, crystal quality data by any other measurement methods may be used. They only have to use, for the evaluation function, the difference between the crystal quality data of an ideal crystal obtained by simulation and the crystal quality data of crystals actually grown.

While in the present embodiment according to the present invention, the case using a regression curve as machine learning and the case using Bayesian optimization as they are presented, other machine learning may be used.

While in the embodiments according to the present invention, one parameter (time of feeding raw material gas during the growth interruption time) is employed as the crystal growth condition to be optimized, a plurality of parameters may be employed. For example, analysis using machine learning such as an AI (artificial intelligence) including deep machine learning on tutor data, a large amount of data having a structure composed of a plurality of parameters and a plurality of evaluation functions in crystal growth data accumulated for a long period of time, can also be performed to optimize the crystal growth conditions.

An example of the data structure of the crystal growth data in this case includes: the crystal growth condition item IDs indicating the items (parameters) of the crystal growth conditions (corresponding to the PH3 time and the AsH3 time in the present embodiment according to the present invention); the crystal growth condition data indicating the crystal growth condition values at the crystal growth condition item IDs (corresponding to each PH3 time and each AsH3 time set to periods of −5 to 10 seconds in the present embodiment according to the present invention); and the crystal evaluation data which makes a pair together with the crystal growth condition data (corresponding to the EF and the EI for each of the PH3 time and the AsH3 time in the present embodiment according to the present invention).

In the analysis of the crystal growth conditions, first, the crystal growth condition item ID targeted is selected. Next, the evaluation function is calculated from the crystal growth condition data at the selected crystal growth condition item ID and the paired crystal evaluation data. Next, the evaluation function is analyzed by machine learning to analyze the optimized crystal growth conditions. Subsequently, a different crystal growth condition item (parameter) ID is selected to repeat the similar analysis, and thereby, the crystal growth conditions can also be optimized with a plurality of parameters (crystal growth condition items) targeted.

While the embodiments according to the present invention employ superlattice crystals as the objects to be optimized, not limited to these, single-layer crystals may be employed. As to their materials, semiconductor crystals such as GaAs-based crystals, GaN-based crystals and SiGe-based crystals, and otherwise, crystals of dielectrics and the like may be employed as well as InP-based crystals.

While in the embodiments according to the present invention, XRD measurement and PL measurement are used, other measurement for crystal evaluation may be used. Crystallographic measurement methods such as Rutherford backscattering spectroscopy and Raman spectroscopy, light absorption measurement methods, optical measurement methods such as an ellipsometry method, electric measurement methods such as Hall measurement and a capacitance-voltage method, and the like may be used.

### INDUSTRIAL APPLICABILITY

Embodiments of the present invention can be applied to growth of high quality crystals of semiconductor and the like, and these crystals can be applied to various electronic devices and optical devices.

### REFERENCE SIGNS LIST

121 XRD curve (crystal quality data) of an ideal crystal
122 XRD curve (crystal quality data) of a sample crystal
123 Difference between the XRD curve (crystal quality data) of the ideal crystal and the XRD curve (crystal quality data) of the sample crystal

1.-6. (canceled)

7. An analysis method of crystal growth conditions, the analysis method comprising:
calculating an evaluation function based on crystal quality data obtained by measuring crystals grown under different crystal growth conditions;
performing machine learning of the evaluation function; and
obtaining optimum crystal growth conditions from a result of the machine learning.

8. The analysis method according to claim 7, wherein the evaluation function is based on a difference between crystal quality data of an ideal crystal and the crystal quality data of the grown crystals.

9. The analysis method according to claim 7, wherein the evaluation function is based on a ratio between an intensity and a half-value width of a peak of the crystal quality data of the grown crystals.

10. The analysis method according to claim 7, wherein:
the crystal quality data includes an X-ray diffraction curve; and
the evaluation function is based on a ratio between an intensity of a substrate peak and an intensity of a diffraction peak on the X-ray diffraction curve.

11. A non-transitory computer readable storage medium storing an analysis program of crystal growth conditions for causing an analysis system of crystal growth conditions which analyzes optimized crystal growth conditions to function to perform the analysis method of claim 7.

12. An analysis system of crystal growth conditions, the analysis system comprising:
an input device configured to input crystal growth conditions and crystal growth data based on crystal quality data obtained by measuring crystals grown under different crystal growth conditions;
a storage device;
a processor configured to acquire the crystal growth data stored in the storage device, calculate an evaluation function from the crystal growth data, perform machine learning on the crystal growth conditions and the evaluation function, and analyze optimized crystal growth conditions; and
an output device configured to output a result obtained by the analysis.

13. The analysis system according to claim 12, wherein the evaluation function is based on a difference between crystal quality data of an ideal crystal and the crystal quality data of the grown crystals.

14. The analysis system according to claim 12, wherein the evaluation function is based on a ratio between an intensity and a half-value width of a peak of the crystal quality data of the grown crystals.

15. The analysis system according to claim 12, wherein:
the crystal quality data includes an X-ray diffraction curve; and
the evaluation function is based on a ratio between an intensity of a substrate peak and an intensity of a diffraction peak on the X-ray diffraction curve.

16. A data structure of crystal growth data used for an analysis system of crystal growth conditions, the analysis system comprising an input device, a storage device, a processor, and an output device, the data structure of crystal growth data being stored in the storage device, and the data structure comprising:
a crystal growth condition item ID indicating an item of crystal growth conditions;
crystal growth condition data indicating a crystal growth condition value at the crystal growth condition item ID; and
crystal evaluation data which makes a pair together with the crystal growth condition data;
wherein the processor is configured to:
select the crystal growth condition item ID in the storage device;
acquire from the storage device, for the selected crystal growth condition item ID, the crystal growth condition data and the crystal evaluation data that makes the pair together with the crystal growth condition data to calculate an evaluation function;
analyze the evaluation function by machine learning; and
analyze optimized crystal growth conditions.

17. The data structure according to claim 16, wherein the output device is configured to output a result obtained by the analysis.

18. The data structure according to claim 16, wherein the evaluation function is based on a difference between crystal quality data of an ideal crystal and crystal quality data of the grown crystals.

19. The data structure according to claim 16, wherein the evaluation function is based on a ratio between an intensity and a half-value width of a peak of crystal quality data of the grown crystals.
